(19) United States
(12) Patent Application Publication
(10) Pub. No.: US 2022/0327817 A1
(43) Pub. Date: Oct. 13, 2022

(54) METHODS AND SYSTEMS FOR REMOTE IMAGE PROCESSING

(71) Applicant: Hypermed Imaging, Inc., Memphis, TN (US)

(72) Inventor: Mark Anthony Darty, Collierville, TN (US)

(21) Appl. No.: 17/499,929

(22) Filed: Oct. 13, 2021

Related U.S. Application Data
(63) Continuation of application No. 15/878,253, filed on Jan. 23, 2018, now abandoned.

(51) Int. Cl. G06V 10/94; G16H 10/60; G16H 30/20; G16H 30/40; G16H 80/00

(52) U.S. Cl. CPC G06V 10/95; G16H 10/60; G16H 30/20; G16H 30/40; G16H 80/00; G06V 10/58

(57) ABSTRACT

Systems and methods for processing hyperspectral image via a computer network. At a server: obtaining, from a first client in a plurality of clients, a request for a unique patient identifier; in response to the request, generating the unique patient identifier independent of patient identity; storing the unique patient identifier and information identifying the first client in a server lookup table; and transmitting the unique patient identifier to the first client; receiving, from a hyperspectral imaging device, a hyperspectral image data set, which includes including a plurality of sub-images of an object and the unique patient identifier, and the unique patient identifier is associated with the plurality of sub-images; forming a hyperspectral image using the hyperspectral image data set; identifying the first client in the plurality of clients using the unique patient identifier and the server lookup table; and transmitting, to the first client, the hyperspectral image.

### CROSS-REFERENCE TO RELATED APPLICATIONS

This application claims priority to U.S. Provisional Patent Application Ser. No. 61/655,800, filed Jun. 5, 2012, and U.S. Provisional Application Ser. No. 61/715,273, filed Oct. 17, 2012, and U.S. application Ser. No. 15/878,253, filed Jan. 23, 2018, the disclosures of which are hereby incorporated herein by reference in their entireties for all purposes.

### TECHNICAL FIELD

The disclosed implementations relate generally to processing hyperspectral images via a computer network.

### BACKGROUND

Hyperspectral imaging is a powerful technique and has been widely used in a large number of applications, such as medical diagnostics, detection and identification of the surface and atmospheric constituents present, analysis of soil type, monitoring agriculture and forest status, environmental studies, and military surveillance. Hyperspectral images are generated by collecting narrow and contiguous spectral bands of data such that a complete reflectance spectrum can be obtained for each point in the region being viewed by the instrument. As such, Hyperspectral/multispectral spectroscopy is an imaging technique that integrates multiples images of an object resolved at several spectral bands (e.g., narrow ranges of wavelengths) into a single data unit. Data provided by hyperspectral/multispectral spectroscopy allow for the identification of individual components of a complex composition through the recognition of hyperspectral/multispectral signatures for individual components within the single data unit.

Application of hyperspectral/multispectral spectroscopy in a clinical setting, however, has not reached its full potential. For example, instruments for processing hyperspectral images are often costly, due to the complex optics required to resolve images at a variety of narrow spectral bands. Hyperspectral/multispectral imaging can also suffer from poor temporal and spatial resolution, as well as low optical throughput, due largely to the complex optics and taxing computational requirements for assembling, processing, and analyzing data associated with hyperspectral images. Also, legislation and ethical considerations require that hyperspectral images of a patient taken in the clinical setting be processed in such a way that patient privacy is fully protected, and physician-patient conditionality is strictly maintained.

### SUMMARY

The problems with conventional approaches to processing hyperspectral images described above are reduced or eliminated by the disclosed systems and methods described below.

In one aspect, hyperspectral/multispectral imaging devices and accompanying methods are provided which allow for both spectral and spatial resolution at low cost, owing to the division of hardware and computational requirements for imaging (e.g., with a low cost hyperspectral/multispectral camera) and image processing (e.g., using one or more remote servers).

In many situations, it is advantageous to provide systems and methods that provide more computational power to process hyperspectral images, without breaching patient confidentiality (e.g., patient privacy or anonymity). In particular, an approach that associates a unique patient identifier with a hyperspectral image of a patient, and transmits the hyperspectral image for processing by a remote server—that has no knowledge of patient identity—via a computer network (e.g., a cloud), can process hyperspectral images generated in the clinical setting more efficiently, without the attendant costs of putting private patient information at risk.

In some embodiments, a method is performed at a server system having one or more processors and memory storing one or more programs for execution by the one or more processors so as to perform the method. The method includes obtaining a request for a unique patient identifier from a first client computer in a plurality of client computers. A unique patient identifier is generated independent of patient identity in response to the request. The unique patient identifier and information identifying the first client computer is stored in a server lookup table. The unique patient identifier is also transmitted to the first client computer. The method further includes receiving, from a hyperspectral imaging device, a hyperspectral image data set. The hyperspectral image data set includes a plurality of sub-images of an object and the unique patient identifier. As such, the unique patient identifier is associated with the plurality of sub-images. The method further includes forming a hyperspectral image using the hyperspectral image data set. The first client computer in the plurality of client computers is identified using the unique patient identifier included in the hyperspectral image data set as well as the server lookup table to thereby transmit the hyperspectral image to the first client computer.

In some embodiments, a method is performed at a client system having one or more processors and memory storing one or more programs for execution by the one or more processors so as to perform the method. The method includes requesting a unique patient identifier for a first patient from a server. The server is configured to receive such requests for unique patient identifiers from a plurality of client computers. In response to the request, the unique patient identifier is obtained. This unique patient identifier is independent of identity of the first patient. The method further includes storing information identifying the patient identity and the unique patient identifier in a client lookup table. The method further includes receiving a hyperspectral image from the server. The hyperspectral image is formed from processing a hyperspectral image data set. The hyperspectral image data set includes a plurality of sub-images of an object and is associated with the unique patient identifier. The method further includes associating the hyperspectral image with the first patient using the unique patient identifier and the client lookup table.

In accordance with some embodiments, a computer system (e.g., a client system or a server system) includes one or more processors, memory, and one or more programs. The one or more programs are stored in memory and are configured to be executed by the one or more processors and the one or more programs include instructions for performing the operations of the methods described above. In accordance with some embodiments, a non-transitory computer readable storage medium has stored therein instructions which when executed by one or more processors, cause a computer system (e.g., a client system or a server system) to perform the operations of the methods described above.

### BRIEF DESCRIPTION OF THE DRAWINGS

The implementations disclosed herein are illustrated by way of example, and not by way of limitation, in the figures of the accompanying drawings. Like reference numerals refer to corresponding parts throughout the drawings.

FIG. 1 is a block diagram illustrating a distributed client-server system, in accordance with some implementations.

FIG. 2 is a block diagram illustrating a client system, in accordance with some implementations.

FIG. 3 is a block diagram illustrating a server system, in accordance with some implementations.

FIG. 4 is a block diagram illustrating an imaging device, in accordance with some implementations.

FIG. 5A is block diagram illustrating an example of a server lookup table, in accordance with some implementations.

FIG. 5B is block diagram illustrating an example of a client lookup table, in accordance with some implementations.

FIG. 5C is block diagram illustrating an example of several hyperspectral image data sets, in accordance with some implementations.

FIG. 6 is a flowchart illustrating a method for processing a hyperspectral image via a computer network in a client-server system, in accordance with some implementations.

FIG. 7 is a flowchart illustrating a method for processing hyperspectral images via a computer network, at a server, in accordance with some implementations.

FIG. 8 is a flowchart illustrating a method for processing hyperspectral images via computer network, at a client, in accordance with some implementations.

### DETAILED DESCRIPTION

The implementations described herein provide techniques for processing hyperspectral images via a computer network.

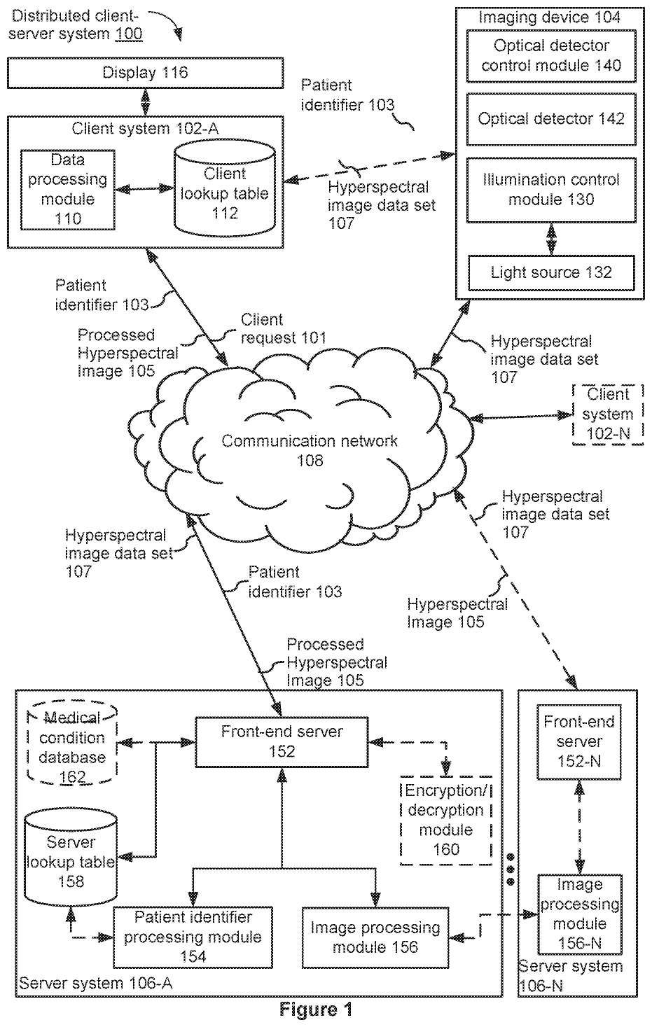

FIG. 1 is a block diagram illustrating a distributed client-server system 100 for processing hyperspectral images via a computer network, in accordance with some implementations.

In some implementations, the distributed client-server system 100 includes one or more client systems 102 (“clients 102,” e.g., client 102-A . . . client 102-N), an imaging device 104, one or more server systems 106 (“servers 106,” e.g., server 106-A . . . server 106-N), and a communication network 108.

In some implementations, the client 102 sends a client request (e.g., a client request 101), requesting a unique patient identifier from the server system 106. In some implementations, after sending the client request, the client 102 receives a unique patient identifier (e.g., a patient identifier 103) from the server 106 (via communication network 108). In some implementations, the client 102 receives a processed hyperspectral image (e.g., a processed hyperspectral image 105) from the server 106.

In one embodiment, a clinician, or medical establishment (e.g., clinic, hospital, HMO, or PPO) with which the clinician is associated, purchases a limited or unlimited-use contract for processing of a plurality of hyperspectral image data sets over a given period of time (e.g., 1 month, 2 months, 3 months, 4 months, 6 months, 12 months, or 24 months). For example, in one implementation, a clinician or medical establishment purchases a license granting use of an unlimited number of processing events over a twelve month period. In another implementation, a clinician or medical establishment pre-purchases a set number of processing events at a pre-determined price (e.g., 100 processing events at X dollars each).

In other embodiments, the clinician or medical establishment is charged on a per use basis, e.g., is charged every time a processing event is requested. For example, the clinician or medical establishment requesting processing of a hyperspectral image data set is charged a one-time fee for processing of the unique data set. Accordingly, in some implementations, in response to receiving a request for a unique patient identifier, or upon receiving a hyperspectral image data set 107, from the imaging device 104, the server 106 sends a fee request to the client 102 (e.g., an invoice) or a credit source associated with the client 102 (e.g., a credit card, PAYPAL, etc.). In one implementation, where a clinician or medical establishment is billed on a fixed schedule, e.g., monthly or bi-annually, in response to receiving a request for a unique patient identifier, or upon receiving a hyperspectral image data set 107, from the imaging device 104, the server 106 creates a billing event on an internal log, which is included on a fee request (e.g., invoice) at the pre-determined time of billing.

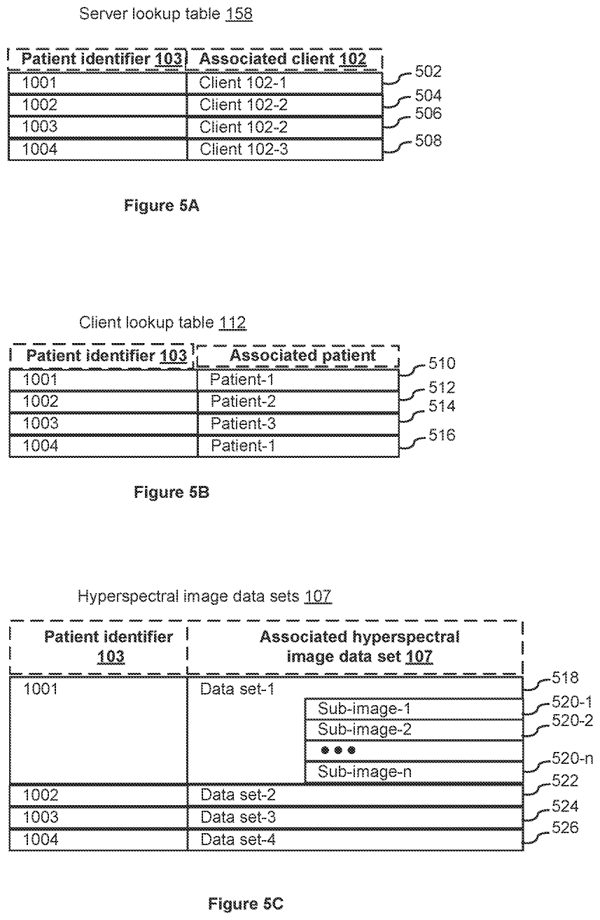

In some implementations, the client 102 includes a data processing module 110, and a client lookup table 112. In some implementations, the data processing module 110 associates a processed hyperspectral image 105 with patient identity in accordance with the client lookup table 112. In some implementations, the data processing module 110 provides additional processing to a processed hyperspectral image received from the server 106, such as image contrast or size adjustment, zoom-in or zoom-out, and rotation. In some implementations, the client lookup table 112 includes patient information (e.g., a patient's name, date of birth, home address, and/or social security number), one or more unique patient identifiers (e.g., a randomly or pseudo-randomly generated identifier that uniquely identifies a patient), and one or more mappings therebetween. More detail about the client lookup table 112 is explained with reference to FIG. 5B. In some implementations, the client 102 is connected to a display 114 (e.g., a computer monitor or projector). In some implementations, the client 102 is a desktop computer, a laptop, a pad type device, or a smart phone.

In some implementations, the imaging device 104 illuminates an object (e.g., an area of a patient's body) and generates imaging data of the object. In some implementations, the imaging device 104 illuminates an object with one or more light sources. In some implementations, after illuminating the object, or concurrently thereto, the imaging device 104 generates and transmits imaging data (e.g., the hyperspectral image data set 107) corresponding to the object to one or more servers 106 for forming a processed hyperspectral image. In other implementations, the imaging device 104 also transmits the hyperspectral image data set 107 to the client 102, where the processed hyperspectral image is formed using the hyperspectral image data set 107. In still other implementations, the imaging device 104 forms the processed hyperspectral image using the hyperspectral image data set 107, and transmits the processed hyperspectral image to the server 106 or the client 102.

In some implementations, the imaging device 104 includes an illumination control module 130 and a light source 132. In some implementations, the light source 132 illuminates a target object (e.g., an area of a patient's body or a body tissue).

In some embodiments, the light source 132 comprises a first incoherent light source. In some implementations, the first incoherent light source is configured to emit near-infrared radiation. In some implementations, the first incoherent light source is a light-emitting diode. In some implementations, the first incoherent light source is a broadband light source, where the broadband light source emits radiation of wavelengths spanning at least 200 nm. In some implementations, the broadband light source emits radiation of wavelengths spanning at least 500 nm. In some implementations, the broadband light source is a white light-emitting diode.

In some embodiments, the light source 132 comprises a plurality of lights (e.g., a first light, and a second light). In some embodiments, each light in the plurality of lights emits a different spectrum of light, e.g., a spectrum of less than 100 nm, less than 200 nm, less than 300 nm, or less than 400 nm. In some embodiments the different spectrums of light emitted by the plurality of lights collectively span the visible light spectrum. In some embodiments the different spectrums of light emitted by the plurality of lights collectively span the visible light spectrum and a portion of the near-infrared spectrum. In some embodiments the different spectrums of light emitted by the plurality of lights collectively span the visible light spectrum and a portion of the ultraviolet spectrum. In some embodiments the different spectrums of light emitted by the plurality of lights collectively span the visible light spectrum, a portion of the ultraviolet spectrum, and a portion of the infrared spectrum. In some embodiments, each light in the plurality of lights emits a different spectrum of light after filtering through one or more band pass filters. In some embodiments, each light in the plurality of lights emits a different spectrum of light without any requirement for filtering through one or more band pass filters. In some embodiments, the imaging device 104 comprises a plurality of light sources 132.

In some implementations, the imaging device 104 includes an optical detector control module 140 and an optical detector 142. In some implementations, the optical detector control module 140 directs the optical detector 142 to collect images (e.g., a plurality of sub-images) of an object for hyperspectral/multispectral analysis after illuminating the object with light in a plurality of different wavelengths. In some implementations, optical detector control module 140 directs the collection of images of an object at two wavelengths: λ and λ′. In some implementations, the illumination control module 130 turns on a first light, emitting light at a first wavelength (λ) and illuminating a region of interest (“ROI”) of the object. After collecting backscattering of the first light from the object, the optical detector 142 forms a first sub-image of the object. In some implementations, after (or while) forming the first sub-image, the illumination control module 130 turns off the first light and turns on a second light, emitting light at a second wavelength (λ′) and illuminating the ROI again. In some implementations, the optical detector control module 140 directs the collection of backscattering of the second light, the illumination control module 130 turns off the second light, and forms a second sub-image of the object.

In some embodiments, more than one optical detector 142 is used to collect sub-images of the object at different wavelengths. In some implementations, a first optical detector 142-1 collects backscattering of the first light having wavelength (λ) to form a first sub-image of the object and a second optical detector 142-2 collects backscattering of the second light having wavelength (λ′) to form a second sub-image of the object. In some embodiments, at least two sub-images are formed concurrently. In a specific embodiment, the illumination control module 130 turns on a first light source emitting light at a first wavelength (λ) and a second light source emitting light at a second wavelength (λ′), such that the ROI is illuminated by light at the first and second wavelengths simultaneously. In some implementations, where at least two sub-images are formed concurrently, each respective sub-image is collected by a different optical detector 142. In other implementations, where at least two sub-images are formed concurrently, each respective sub-image is collected by the same optical detector 142. In some implementations, the above described processes are repeated a predefined number of times with light of a variety of wavelengths or wavelength bands, to generate a plurality of sub-images of the ROI. For instance, in some embodiments, two different wavelengths or wavelength bands are sub-imaged in this manner. In other embodiments, three, four, five, six, seven, eight, nine, ten, eleven, twelve, thirteen, fourteen or fifteen different wavelengths or wavelength bands are sub-imaged in this manner. In certain embodiments, all of the sub-images are collected by the same optical detector 142. In other embodiments, more than one optical detector 142 is used to collect the sub-images. In some embodiments, the optical detector 142 is a charge-coupled device (CCD) array.

In some implementations, after a plurality of sub-images is generated, the imaging device 104 generates imaging data (e.g., the hyperspectral image data set 107) in accordance with the sub-images. In some embodiments the plurality of sub-images is sent to the server system 106 to form an imaging data set and processed hyperspectral image 105. In some embodiments the plurality of sub-images is sent to the client system 102 to form an imaging data set and processed hyperspectral image 105. In some implementations, the hyperspectral image data set 107 prepared by the imaging device 104 is transmitted to one or more servers 106 for further processing (e.g., forming the processed hyperspectral image 105 or comparison to a medical condition database 162). In other implementations, the hyperspectral image 105 is formed by the imaging device 104.

In some implementations, the plurality of sub-images is sent to the client system 102 for pre-processing (e.g., compression of the sub-images, encryption, or creation of the hyperspectral image data set 107). In some implementations, the client system sends the pre-processed sub-images or hyperspectral image data set 107 to one or more servers 106 for further processing (e.g., forming the hyperspectral image 105 or comparison to a medical condition database 162). In certain embodiments, where the imaging device sends the plurality of sub-images to the client system 102-A-1, associated with a first institution, the server 106 transmits processed data (e.g., hyperspectral image 105, a diagnosis, or a prognosis) to a second client system 102-A-2, associated with the same institution.

In some implementations, the server 106 (i) generates a unique patient identifier (e.g., the patient identifier 103) in response to a request from the client 102, and (ii) processes a hyperspectral image data set (e.g., the hyperspectral image data set 107 or sub-images) received from the imaging device 104 to form a hyperspectral image (e.g., the hyperspectral image 105). In some implementations, the server 106 includes a frontend server 152, a patient identifier processing module 154, an imaging processing module 156, a server lookup table 158, optionally, an encryption/decryption module 160, and, optionally, a medical condition database 162. In some implementations, the frontend server 152 receives and relays client requests to corresponding modules within the server 106. In some implementations, the frontend server 152 also transmits the patient identifier 103 and the corresponding processed hyperspectral image 105 to the client 102 for review by a physician. In some implementations, the patient identifier processing module 154 generates a patient identifier 103 in response to a request from the client 102. In some implementations, the patient identifier processing module 154 also stores in the server lookup table 158 (i) the unique patient identifier 103 and (ii) information identifying a specific client (e.g., the client 102-A) among a plurality of clients 102. In some implementations, the imaging processing module 156 processes the hyperspectral image data set 107 received from the imaging device 104 to form a processed hyperspectral image 105.

In some implementations, the server lookup table 158 includes one or more mappings between unique patient identifiers and information identifying respective clients 102. In some implementations, the encryption/decryption module 160 decrypts an encrypted hyperspectral image data set for processing by the image processing module 156, or encrypts a hyperspectral image generated by the image processing module 156 before the hyperspectral image is transmitted to the client 102.

In some implementations, the medical condition database 162 includes one or more hyperspectral signatures, which correspond to one or more medical conditions, for comparison with a processed hyperspectral image 105 generated by the image processing module 156.

In some implementations where the distributed client-server system 100 includes a plurality of clients 102 (e.g., the client 102-A, . . . , and the client 102-N), the server 106 identifies a specific client 102 (e.g., the client 102-A) among a plurality of clients 102, in accordance with the unique patient identifier associated with a hyperspectral image data set, and the server lookup table 158. In some implementations, after the identification process, the server 106 transmits the hyperspectral image formed based on the hyperspectral image data set, to the identified client 102. In some implementations, after the identification process, the server 106 transmits a diagnosis, probability of each of one or more diagnosis, or likelihood or odds of each of one or more diagnosis, based on a comparison between the hyperspectral image data set 107 or processed hyperspectral image 105 and the medical condition database 162, to the identified client 102. In yet other implementations, the server 106 transmits a processed hyperspectral image 105 and a diagnosis, probability of one or more diagnosis, or prognosis for one or more treatments to the identified client 102. In other implementations, the server 106 transmits the processed hyperspectral image 105 to one or more clients 102 that share a predefined relationship with the identified client 102. For example, in some implementations, the server 106 transmits the processed hyperspectral image 105 to clients 102 that are within a same local area network (e.g., an internal network owned by a same hospital or clinic) as the identified client 102. As another example, in some implementations, the server 106 transmits the processed hyperspectral image 105 to clients 102 that are registered as belonging to a same physician as the identified client 102. As still another example, in some implementations, the server 106 transmits the hyperspectral image to clients 102 that belong to person given authorization by the treating physician to review the hyperspectral image, such as physician assistants who work under the supervision of the treating physician. This approach provides flexibility but still protects patient privacy. In this approach, a hyperspectral image 105 indicative of a patient's medical condition is sent to one or more computer, when these computers are under control of a same treating physician, thereby minimizing the risk of breaching patient confidentiality by an unauthorized third party.

In some implementations where the distributed client-server system 100 includes a plurality of servers 106 (e.g., the server 106-A, . . . , and the server 106-N), one server 106 (e.g., the server 106-A) communicates with another server 106 (e.g., the server 106-N). In some implementations, the image processing module 156 of one server 106 communicates with the image processing module 156 of another server 106. In some implementations, the imaging device 104 transmits different portions of a hyperspectral image data set to different servers 106—e.g., a first portion of the hyperspectral image data set 107 to the server 106-A, and a second portion (e.g., different from the first portion) of the hyperspectral image data set 107 to the server 106-N—for parallel processing. In some implementations, where redundancy is desired, the imaging device 104 transmits the same portion of a hyperspectral image data set to two or more servers 106 (with different computational power)—e.g., the same portion of the hyperspectral image data set 107 to both the server 106-A and the server 106-N—and results from the server 106 that finishes the processing earliest are used to form the hyperspectral image.

In some implementations, the communication network 108 optionally includes the Internet, one or more location connections, one or more local area networks (LANs), one or more wide area networks (WANs), other types of networks, or a combination of such networks. In some implementations, the one or more location connections optionally include connections by infrared signals, radio frequency signals, local area networks (LANs), Bluetooth, serial or parallel cable, or a combination of thereof. In some embodiments, the imaging device 104 is connected to the communication network 108 through a wireless 802.11 device. In some embodiments, the imaging device 104 is in direct electronic communication with the system server 106 through a wireless 802.11 device. In such instances, the wireless 802.11 device is construed as the communication network 108.

In some embodiments, the imaging device 104 includes a docking port and is configured to be docked directly onto the server system 106. In such embodiments, the docking interface between the imaging device 104 and the server system 106 is construed as the communication network 108.

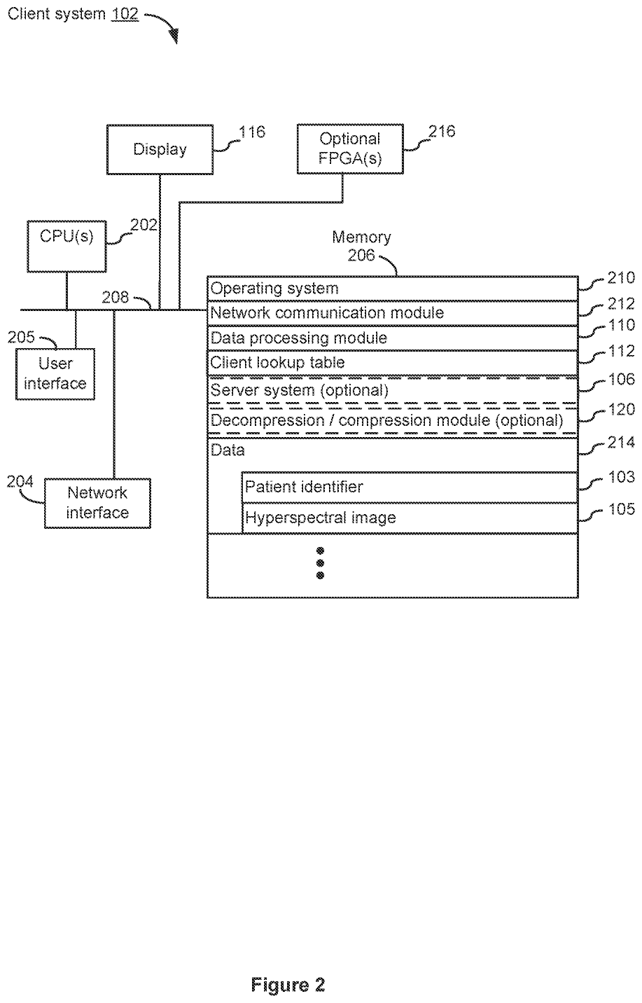

FIG. 2 is a block diagram illustrating a client system 102 (“client 102”) for processing hyperspectral images via a computer network, in accordance with some implementations.

In some implementations, the client 102 includes one or more processing units CPU(s) 202 (also called processors), one or more network or other network interfaces 204, a user interface 205, memory 206, a display 116, one or more optional floating point gate arrays (FPGAs) 216 and one or more communication buses 208 for interconnecting these components. In some embodiments, the one or more optional FPGAs 216 are used to compress or decompress hyperspectral information (e.g., hyperspectral image data sets 107 and/or processed hyperspectral images 105) using lossy or lossless compression. Exemplary compression techniques include, but are not limited to, vector quantization (VQ) based algorithms such as mean-normalized vector quantization (M-NVQ). See Ryan and Arnold, “The lossless compression of AVIRIS images by vector quantization,” IEEE Trans. Geoscience and Remote Sensing, Vol. 35, No. 3, May 1997, which is hereby incorporated herein by reference. Exemplary compression techniques that may be encoded by optional FPGAs 216 further include, but are not limited to, VQ based algorithms that involve locally optimal design of a partitioned vector quantizer for the encoding of source vectors drawn from hyperspectral images. See Motta et al., “Compression of hyperspectral imagery,” Data Compression Conference. Proceedings. DCC 2003, pp. 25-27, March 2003 which is hereby incorporated herein by reference. Exemplary compression techniques that may be encoded by optional FPGAs 216 further include, but are not limited to, spatial M-NVQ techniques that jointly optimized the spatial M-NVQ and spectral Discrete Cosine Transform (DCT) to produce compression ratios significantly better than those obtained by the optimized spatial M-NVQ technique alone. See Pickering and Ryan, “Efficient spatial-spectral compression of hyperspectral data,” IEEE Trans. Geoscience and Remote Sensing, 39, No. 7, July 2001, which is hereby incorporated herein by reference. Exemplary compression techniques that may be encoded by optional FPGAs 216 further include, but are not limited to, wavelet transform algorithms such as Shapiro's embedded zerotree wavelet, set partitioning in hierarchical trees, and set partitioned embedded bloCK. See Shapiro, “Embedded image coding using zerotrees of wavelet coefficients,” IEEE Trans. Signal Processing, vol. 41, pp. 3445-3462, December 1993, Said and Pearlman, “A new, fast and efficient image codec based on set partitioning in hierarchical trees,” IEEE Trans. on Circuits and Systems for Video Technology 6, pp. 243-250, June 1996, and Islam and Pearlman, “An embedded and efficient low-complexity hierarchical image coder,” in Proc. SPIE Visual Comm. and Image Processing, vol. 3653, pp. 294-305, 1999. In some implementations, the user interface 205 includes a keyboard, mouse, touchpad, touchscreen or other input device. The communication buses 208 optionally include circuitry (sometimes called a chipset) that interconnects and controls communications between system components. The memory 206 typically includes high-speed random access memory, such as DRAM, SRAM, DDR RAM or other random access solid state memory devices; and optionally includes non-volatile memory, such as one or more magnetic disk storage devices, optical disk storage devices, flash memory devices, or other non-volatile solid state storage devices. The memory 206 optionally includes one or more storage devices remotely located from the CPU(s) 202. The memory 206, or alternatively the non-volatile memory device(s) within the memory 206, comprises a non-transitory computer readable storage medium. In some implementations, the memory 206 or alternatively the non-transitory computer readable storage medium stores the following programs, modules and data structures, or a subset thereof:
an operating system 210, which includes procedures for handling various basic system services and for performing hardware dependent tasks;
a network communication module (or instructions) 212 for connecting the client 102 with other devices (e.g., the imaging device 104 or the server 106) via one or more network interfaces 204 (wired or wireless), and the communication network 108 (FIG. 1);
a data processing module 110 for generating a request for a unique patient identifier, and for processing hyperspectral image data sets or hyperspectral images received from other devices (e.g., the server 106 or the imaging device 104);
a client lookup table 112 for including unique patient identifiers, patient identifying information, and mappings therebetween (see FIG. 5B);
optionally, the server 106, implemented in hardware (e.g., IC chips) or software (e.g., programming packages);
optionally, a decompression/compression module 120, using CPU(s) and/or optional FPGA(s) to compress or decompress hyperspectral information (e.g., hyperspectral image data sets 107 and/or processed hyperspectral images 105) using a lossy or a lossless compression technique such as any of the aforementioned compression techniques described above with respect to optional FPGA(s); and
data 214 stored on the client 102 or electronically accessible by the client, which includes:
   a patient identifier 103 for identifying, directly or indirectly, a patient; and
   a hyperspectral image 105 indicative of one or more medical conditions (or lack thereof), associated with a patient.

In some implementations, one or more of the above identified elements are stored in one or more of the previously mentioned memory devices, and correspond to a set of instructions for performing a function described above. The above identified modules or programs (e.g., sets of instructions) need not be implemented as separate software programs, procedures or modules, and thus various subsets of these modules may be combined or otherwise re-arranged in various implementations. In some implementations, the memory 206 optionally stores a subset of the modules and data structures identified above. Furthermore, the memory 206 may store additional modules and data structures not described above.

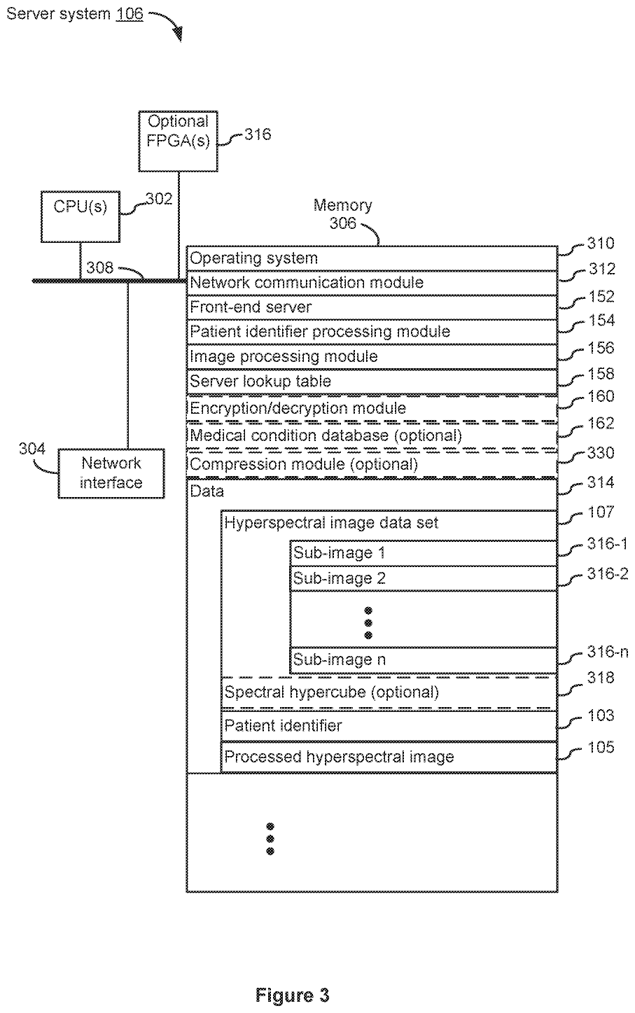

FIG. 3 is a block diagram illustrating a server system 106 (“server 106”) for processing hyperspectral images via a computer network, in accordance with some implementations.

In some implementations, the server 106 includes one or more processing units CPU(s) 302 (also herein referred to as processors), one or more network interfaces 304, memory 306, one or more optional floating point gate arrays (FPGAs) 316, and one or more communication buses 308 for interconnecting these components. The communication buses 308 optionally include circuitry (sometimes called a chipset) that interconnects and controls communications between system components. In some embodiments, the one or more optional FPGAs 316 are used to compress or decompress hyperspectral information (e.g., hyperspectral image data sets 107 and/or processed hyperspectral images 105) using lossy or lossless compression, such as any of the techniques described above in conjunction with FIG. 2 with reference to client 102. The memory 306 includes high-speed random access memory, such as DRAM, SRAM, DDR RAM or other random access solid state memory devices; and optionally includes non-volatile memory, such as one or more magnetic disk storage devices, optical disk storage devices, flash memory devices, or other non-volatile solid state storage devices. The memory 306 optionally includes one or more storage devices remotely located from CPU(s) 302. The memory 306, or alternatively the non-volatile memory device(s) within the memory 306, comprises a non-transitory computer readable storage medium. In some implementations, the memory 306 or alternatively the non-transitory computer readable storage medium stores the following programs, modules and data structures, or a subset thereof:
an operating system 310, which includes procedures for handling various basic system services and for performing hardware dependent tasks;
a network communication module (or instructions) 312 for connecting the server 106 with other devices (e.g., the client 102 or the imaging device 104) via one or more network interfaces 304 (wired or wireless), and the communication network 108 (FIG. 1);
a front-end server 152 for processing incoming client requests (e.g., request for unique patient identifier), and for receiving and relaying hyperspectral image data sets received from the image device 104 to appropriate modules in the server 106;
a patient identifier processing module 154 for generating patient identifiers, storing mappings between patient identifiers and patient identifying information in the server lookup table 158, and associating a patient identifier with a hyperspectral image before its transmission to the client 102;
an imaging processing module 156 for forming a hyperspectral image using a hyperspectral image data set;
a server lookup table 158 for including mappings between unique patient identifiers and respective patient identifying information (see FIG. 5A);
optionally, an encryption/decryption module 160 for decrypting an encrypted hyperspectral image data set 107, or for encrypting a processed hyperspectral image 105 before its transmission to other devices (e.g., the client 102);
optionally, a compression module 330 for compressing hyperspectral information (e.g., hyperspectral image data sets 107 and/or processed hyperspectral images 105), using lossy or lossless compression, such as any of the compression techniques described above in conjunction with FIG. 2 with reference to client 102, optionally in conjunction with, or instead of, use of the optional one or more FPGAs 316;
optionally, a medical condition database 162 for including information concerning one or more medical conditions, and associated symptoms or signatures that are identifiable in a hyperspectral image data set or hyperspectral image; and
data 314 stored on the server 106, which includes:
   a hyperspectral image data set 107 having one or more sub-images of an object and related metadata, including
      one or more sub-images 316-i (e.g., sub-image 316-1, sub-image 316-2 . . . , and sub-image 316-n) of the object and metadata;
   optionally, a spectral hypercube 318 having information generated by assembling sub-images 316-i in accordance with metadata;
   a patient identifier 103 for identifying, directly or indirectly, a patient; and
   a processed hyperspectral image 105 formed using the hyperspectral image data set 107, and indicative of one or more medical conditions (or lack thereof) associated with a patient.

In some implementations, one or more of the above identified elements are stored in one or more of the previously mentioned memory devices, and corresponds to a set of instructions for performing a function described above. The above identified modules or programs (e.g., sets of instructions) need not be implemented as separate software programs, procedures or modules, and thus various subsets of these modules may be combined or otherwise re-arranged in various implementations. In some implementations, the memory 306 optionally stores a subset of the modules and data structures identified above. Furthermore, memory 306 optionally stores additional modules and data structures not described above.

Although FIG. 3 shows a “server system 106,” also referred to as a server, FIG. 3 is intended more as functional description of the various features which may be present in a backend system than as a structural schematic of the implementations described herein. In practice, and as recognized by those of ordinary skill in the art, items shown separately could be combined and some items could be separated.

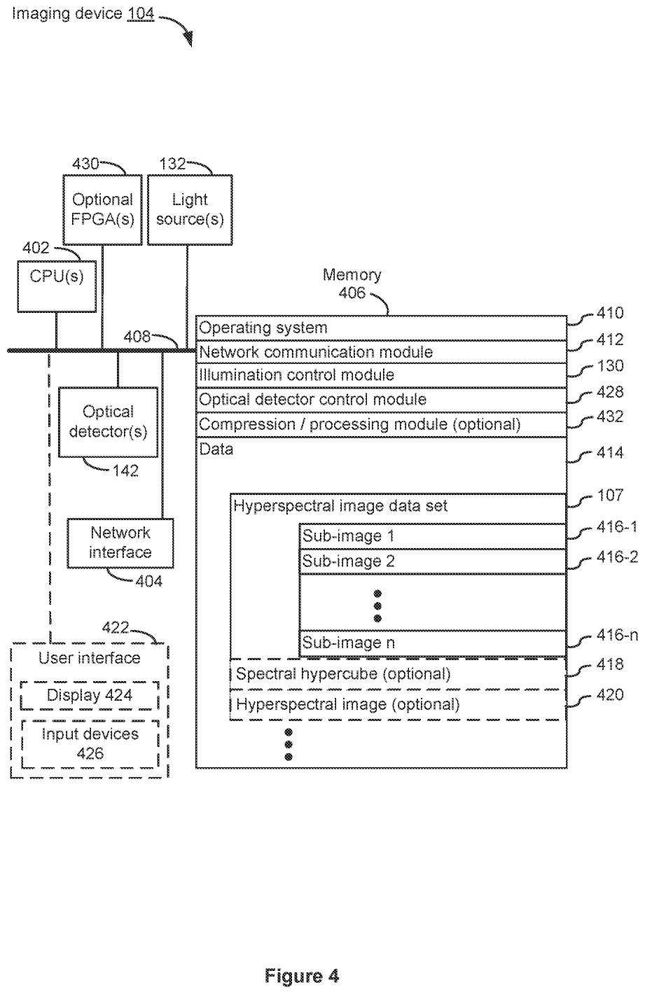

FIG. 4 is a block diagram illustrating an imaging device 104 for processing hyperspectral images via a computer network, in accordance with some implementations.

In some implementations, the imaging device 104 includes one or more processing units CPU(s) 402 (also herein referred to as processors), one or more light sources 132, one or more optical detectors 142, one or more optional floating point gate arrays (FPGAs) 430, one or more network interfaces 404, memory 406, optionally a user interface 422, and one or more communication buses 408 for interconnecting these components. In some implementations, the user interface 422 includes a display 424 (e.g., a LCD), and an input device 426 (e.g., a keyboard or a touchscreen). The communication buses 408 optionally include circuitry (sometimes called a chipset) that interconnects and controls communications between system components. In some embodiments, the one or more optional FPGAs 430 are used to compress or decompress hyperspectral information (e.g., hyperspectral image data sets 107 and/or processed hyperspectral images 105) using lossy or lossless compression, such as any of the techniques described above in conjunction with FIG. 2 with reference to client 102. The memory 406 includes high-speed random access memory, such as DRAM, SRAM, DDR RAM or other random access solid state memory devices; and optionally includes non-volatile memory, such as one or more magnetic disk storage devices, optical disk storage devices, flash memory devices, or other non-volatile solid state storage devices. The memory 406 optionally includes one or more storage devices remotely located from CPU(s) 402. The memory 406, or alternatively the non-volatile memory device(s) within the memory 406, comprises a non-transitory computer readable storage medium. In some implementations, the memory 406 or alternatively the non-transitory computer readable storage medium stores the following programs, modules and data structures, or a subset thereof:
an operating system 410, which includes procedures for handling various basic system services and for performing hardware dependent tasks;
a network communication module (or instructions) 412 for connecting the imaging device 104 with other devices (e.g., the client 102 or the server 106) via one or more network interfaces 404 (wired or wireless), and the communication network 110 (FIG. 1);
an illumination control module 130 for directing the one or more light sources 132 to illuminate an object with light of a plurality of different wavelengths or wavelength bands;
an optical detector control module 428 for directing the one or more optical detectors 142 to collect images (e.g., sub-image(s) 416-i) of an object for hyperspectral/multispectral analysis;
optionally, a compression/processing module 432 for compressing hyperspectral information or otherwise processing the hyperspectral information (e.g., hyperspectral image data sets 107 and/or processed hyperspectral images 105) using, for example, lossy or lossless compression, such as any of the compression techniques described above in conjunction with FIG. 2 with reference to client 102, optionally in conjunction with, or instead of, use of the optional one or more FPGAs 430;
data 414 stored on the imaging device 104, which includes:
   a hyperspectral image data set 107 for including one or more sub-images of an object and related metadata, including
      one or more sub-image(s) 416-i (e.g., sub-image 416-1, sub-image 416-2 . . . , and sub-image 416-n) of the object and its related metadata;
   optionally, a spectral hypercube 418 for including information generated from assembling sub-images 416-i in accordance with metadata; and
   optionally, a hyperspectral image 420 formed using the hyperspectral image data set 107, and indicative of one or more media conditions (or lack thereof), associated with a patient.

In some implementations, one or more of the above identified elements are stored in one or more of the previously mentioned memory devices, and corresponds to a set of instructions for performing a function described above. The above identified modules or programs (e.g., sets of instructions) need not be implemented as separate software programs, procedures or modules, and thus various subsets of these modules may be combined or otherwise re-arranged in various implementations. In some implementations, the memory 406 optionally stores a subset of the modules and data structures identified above. Furthermore, memory 406 optionally stores additional modules and data structures not described above.

FIG. 5A is block diagram illustrating an example of the server lookup table 158 in accordance with some implementations.

In some implementations, the server lookup table 158 includes one or more patient identifiers 103, information identifying one or more clients 102, and mappings therebetween. In some situations, one patient identifier 103 is associated with one client 102. For example, the patient identifier 1001 (502) is associated with the client 102-1. In other situations, for example, where different patients are being treated by a same physician, two patient identifiers 103 are associated with one same client 102. In FIG. 5A, both the patient identifier 1002 (502) and the patient identifier 1003 (503) are associated with the client 102-2, e.g., a computer belonged to a physician who is treating both patients.

In some implementations, the server 106 compares the patient identifier associated with a hyperspectral image data set with patient identifiers stored in the server lookup table 108 to identify a specific client 102 to which a hyperspectral image is to be transmitted. The server 106 then transmits the hyperspectral image to the identified client 102. In other implementations, the server 106 also transmits the hyperspectral image to clients 102 that share a predefined relationship with the identified client 102 (e.g., computers under the control of the same physician).

FIG. 5B is block diagram illustrating an example of the client lookup table 112, in accordance with some implementations.

In some implementations, the client lookup table 112 includes one or more patient identifiers 103, information identifying one or more patients, and mappings therebetween. In some situations, information identifying a patient (e.g., a patient's SSN) is associated with one patient identifier 103. For example, the patient-2 (512) is associated with the patient identifier 1002. In other situations, information identifying a patient (e.g., a patient's SSN) is associated with two or more patient identifiers, for example, when the same patient is being treated by two different physicians connected to the server 106, each of whom assigns a different patient identifier to the patient. For example, patient-1 is associated with both patient identifier 1001 (e.g., by physician 1) and patient identifier 1004 (by physician 2).

In some implementations, the client 102 compares the patient identifier associated with a hyperspectral image with patient identifiers stored in the client lookup table 102 to identify a patient associated with the hyperspectral image (e.g., the patient from whom the hyperspectral image is taken). After the patient is identified, a physician can diagnose the patient in accordance with the associated hyperspectral image.

FIG. 5C is block diagram illustrating an example of several hyperspectral image data sets 107, in accordance with some implementations.

In some implementations, a hyperspectral image data set 107 includes patient identifiers 103, information identifying the hyperspectral image data set, and mappings therebetween. In some implementations, a data set includes a plurality of sub-images. For example, the data set-1 (518) includes n sub-images: sub-image-1 (502-1), sub-image-2 (502-2), through and including sub-image-n (502-n). In some implementations, one hyperspectral data set is associated with one unique patient identifier 103. For example, the data set-1 (518) is associated with one patient identifier (1001).

In some implementations, after receiving a hyperspectral image data set, the server 106 forms a hyperspectral image in accordance therewith. In some implementations, server 106 also associates a hyperspectral image data set and a hyperspectral image formed in accordance therewith with the same patient identifier. For example, after forming the hyperspectral image 105 in accordance with the hyperspectral image data set 107, the server 106 associates the hyperspectral image 105 with the patient identifier 1001, the patient identifier associated with the hyperspectral image data set 107. Using the patient identifier associated with a hyperspectral image, the client 102 can then identify which patient the hyperspectral image is associated.

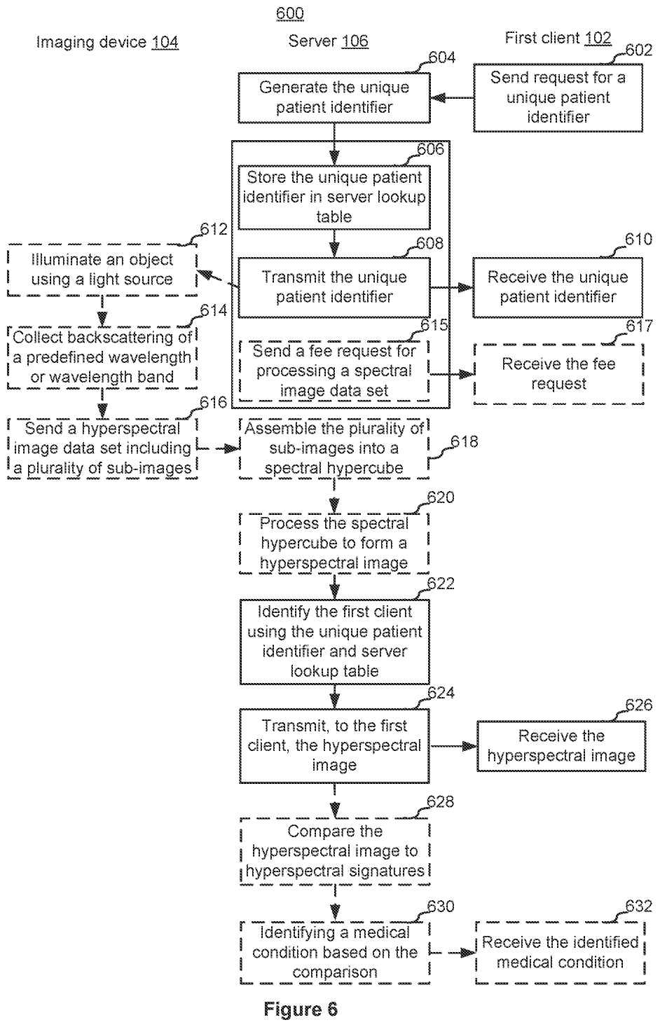

FIG. 6 is a flow chart illustrating a method for processing a hyperspectral image via computer network in a client-server system, in accordance with some implementations of the present disclosure.

In some implementations, to protect patient privacy, before taking a hyperspectral image of a patient, a physician first obtains a unique patient identifier for the patient. In some implementations, to obtain the unique patient identifier, a first client 102 (e.g., a computer being used by the physician) sends (602) a request for a unique patient identifier to the server 106. In some implementations, after receiving the request from the client 102, the server 106 generates (604) the unique patient identifier, and stores (606) the unique patient identifier and information identifying the first client 102 (e.g., the IP address or MAC address associated with the first client 102) in the server lookup table 158. In some implementations, the server 106 then transmits (608) the unique patient identifier back to the first client 102. In some implementations, after receiving (610) the unique patient identifier, the first client 102 stores the unique patient identifier along with the information identifying a patient (for which the identifier is requested) in the client lookup table 112. In some implementations, the server 106 also transmits (608) the unique patient identifier to the imaging device 104. In some embodiments, this unique patent identifier may be reused for the patient when the patient obtains subsequent hyperspectral images over time. In some embodiments, a unique patent identifier must be obtained for a patient each time the patient obtains a hyperspectral image, even when it is the case that one or more other patient identifiers have been issued to the patient for other hyperspectral images in the past.

In some implementations, after receiving the request for a unique patient identifier (602), the server 106 sends (615) a fee request (e.g., an invoice) for processing a hyperspectral image data set to the client 102 or a credit source associated with the client 102 (e.g., a credit card). The client 102 receives (617) the fee request and subsequently remits payment for the requested processing event. In certain implementations, the server sends (615) a fee request for processing a hyperspectral image data set in response to a different step of method 600, for example in response to the imaging device 104 or client 102 sending (616) a hyperspectral image data set including a plurality of sub images to the server 106, upon transmitting (624) the hyperspectral image to the client 102, or upon identifying (630) a medical condition based on the comparison of a hyperspectral image data set or hyperspectral image to hyperspectral signatures.

In one implementation, where a clinician or medical establishment is billed on a fixed schedule, e.g., monthly or bi-annually, in response to receiving a request for a unique patient identifier, or upon receiving a hyperspectral image data set, from the client 102, the server 106 creates a billing event on an internal log, which is included on a fee request (e.g., invoice) at the pre-determined time of billing.

In some implementations, after receiving the unique patient identifier, the imaging device 104 begins the process (612, 614, and 614) of generating a hyperspectral image data set of an object (e.g., an area of the patient's body). In other implementations, the imaging device 104 begins the process (612, 614, and 614) before receiving the unique patient identifier from the server 106. For example, in some implementations, the imaging device 104 begins to obtain the hyperspectral image data set while the server 106 is still generating the unique patient identifier. In some implementations, the server 106 transmits to unique patient identifier to the imaging device 104 before the imaging device 104 transmits the hyperspectral image data set to the server 106 (e.g., when the hyperspectral image data set is still being generated by the imaging device 104). In many situations, this approach is beneficial because it allows the process of obtaining the unique patient identifier at the server 106 to run concurrently with the process of obtaining the hyperspectral image of the object at the imaging device 104.

In some implementations, the imaging device 104 generates the hyperspectral image data set of the object by: (1) illuminating (612) the object using a light source, (2) collecting (614) backscattering of a predefined wavelength or wavelength band to form one or more sub-images of the object, and (3) sending (616) a hyperspectral image data set, which includes a plurality of sub-images, to the server 106, which forms a hyperspectral image using the data set. It will be appreciated that the collecting step 614, in some embodiments, involves collecting the backscattering of a first predefined wavelength or wavelength band to form a first sub-image, and then repeating this collecting up to N times for each predefined wavelength or wavelength band in a plurality of wavelength bands to thereby form a plurality of sub-images, each collected at a different wavelength or wavelength band, where the plurality of sub-images collectively represent the exposure of the same portion of the patient to all or a portion of the visible wavelength spectrum, a portion of the ultraviolet wavelength spectrum a portion of the infrared wavelength spectrum, or some combination thereof. In some implementations, before sending the data set to the server 106, the imaging device 104 associates the unique patient identifier with the data set (FIG. 5C). Alternatively, in other implementations, after generating the hyperspectral image data set, the imaging device 104 also forms a hyperspectral image of the object using the data set, and transmits the hyperspectral image to the server 106, or to the client 102.

In certain implementations, after generating the hyperspectral image data set, the imaging device 104 performs at least one pre-processing function on the hyperspectral image data set, prior to transmitting the data set to the server 106. Non-limiting examples of pre-processing functions include: (i) adjusting brightness of at least one of the sub-images, (ii) adjusting contrast of at least one sub-image, (iii) removing an artifact from at least one sub-image, (iv) cropping at least one sub-image, (v) processing one or more sub-pixels of at least one sub-image, (vi) compressing the size of at least one sub-image, (vii) assembling the plurality of sub-images into a spectral hypercube, (viii) transforming the spectral hypercube, (ix) formatting data contained within at least one sub-image, or (x) encrypting data contained within at least one sub-image. In some embodiments, any of these pre-processing functions are performed using optional compression/processing module 432 and/or optional FPGA(s) 430 depicted in FIG. 4. In certain embodiments, the plurality of sub-images or hyperspectral image data set is transmitted to client 102, which performs one or more pre-processing functions, prior to transmitting to the server 106.

In some implementations, after receiving the hyperspectral image data set, the server 106 begins the process of forming a hyperspectral image in accordance therewith. In some implementations, the server 106 first assembles (618) the plurality of sub-images into a spectral hypercube. In some implementations, the server 106 then processes (620) the spectral hypercube to form a hyperspectral image of the object. In some implementations, forming the hyperspectral image of the object includes at least one of: (i) adjusting brightness of at least one of the sub-images, (ii) adjusting contrast of at least one sub-image, (iii) removing an artifact from at least one sub-image, (iv) cropping at least one sub-image, (v) processing one or more sub-pixels of at least one sub-image, (vi) compressing the size of at least one sub-image, (vii) assembling the plurality of sub-images into a spectral hypercube, (viii) transforming the spectral hypercube, (ix) formatting data contained within at least one sub-image, or (x) encrypting data contained within at least one sub-image.

In some implementations, after forming the hyperspectral image, the server 106 begins the process of transmitting the hyperspectral image to a specific client 102—the first client 102 (e.g., a computer associated with the patient's treating physician)—among a plurality of clients 102. In some implementations, the server 106 identifies (622) the specific client 102 among the plurality of clients 102 (e.g., computers associated with different physicians, including physicians who do not currently have a physician-patient relationship) by comparing (i) the unique patient identifier associated with the hyperspectral image with (ii) the patient identifiers stored in the server lookup table 158 (FIG. 5A).

In some implementations, after identifying the first client 102, the server 106 transmits (624) the hyperspectral image to the first client (e.g., for review by the treating physician). In some implementations, the first client 102 receives (626) the hyperspectral image, and optionally displays the image for a physician to review.

In some implementations, the server 106 further compares (628) the hyperspectral image data set or hyperspectral image with one or more hyperspectral signatures stored on the server 106 (e.g., in the medical condition database 162). In some implementations, the hyperspectral signatures correspond to one or more medical conditions (e.g., ulcer formation and melanoma). In some implementations, a hyperspectral signature includes a set of spectral characteristics unique to a medical condition.

In some implementations, based on the comparison (628), the server 106 identifies one or more medical conditions associated with the patient. In some implementations, based on a degree of similarity between the hyperspectral image data set or hyperspectral image and a hyperspectral signature, the server 106 also determines a probability or likelihood that the patient has an identified medical condition. In some implementations, based on the comparison (628), the server 106 also identifies additional information about a medical condition, e.g., information about whether the condition is malignant or benign, options for treatment, or a prognosis for a treatment.

In certain embodiments, methods are provided for monitoring a medical condition or course of treatment over time. In some implementations, the server 106 compares the hyperspectral/multispectral data set, or hyperspectral image generated therefrom, to a hyperspectral/multispectral data set, or hyperspectral image generated therefrom, acquired at an earlier time from the same patient. In some implementations, based on the comparison of the hyperspectral image data sets acquired at different time points from the same patient, the server 106 also determines a change in an identified medical condition, e.g., progression of a medical condition, regression of a medical condition, or options for continuing, modifying, or abandoning treatment.

In some implementations, after identifying one or more medical conditions based on the comparison, the server 106 transmits the information concerning the identified medical conditions to the first client 102, which receives (632) and displays the information for the treating physician's review.

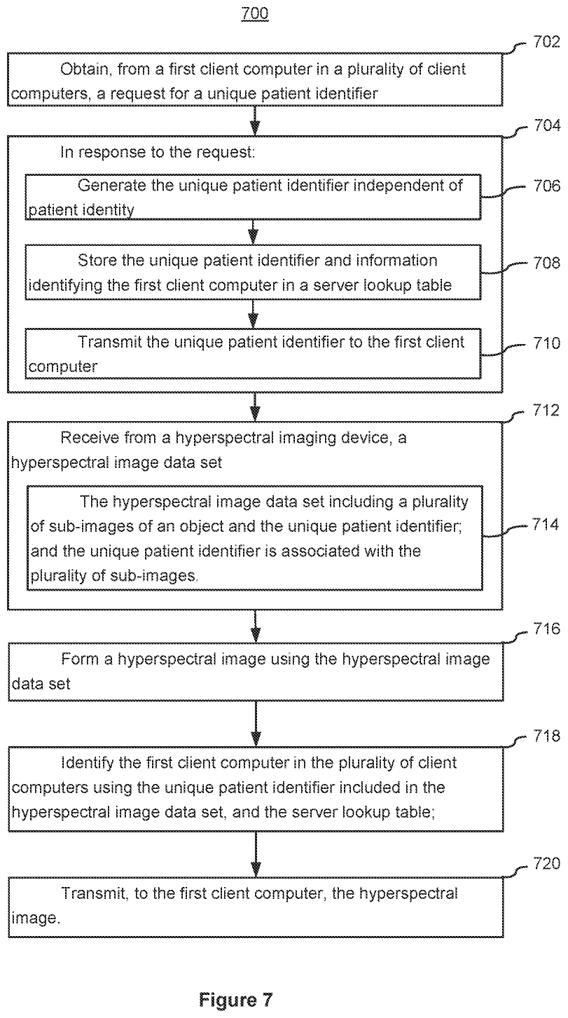

FIG. 7 is a flowchart illustrating a method for processing hyperspectral images via a computer network, at a server, in accordance with some implementations.

In some implementations, a server (e.g., the server 106) obtains (702), from a first client computer (e.g., the client 102-A), a request for a unique patient identifier. In some implementations, the server is configured to receive requests for unique patient identifiers from a plurality of client computers (e.g., the client 102-A . . . the client 102-N). For example, the server 106-A, a server located in a hospital, is communicatively connected with and configured to receive requests for patient identifiers from two or more computers (e.g., computers belong to different physicians or located at different locations).

In response to the request (704), the server generates (706) the unique patient identifier independent of patient identity, stores (708) the unique patient identifier and information identifying the first client computer in a server lookup table (e.g., the server lookup table 158), and transmits (710) the unique patient identifier to the first client computer.

In some implementations, the unique patient identifier is independent of the identity of the first patient. In some implementations, generating the unique patient identifier independently of patient identify includes: generating the unique patient identifier without reference to information relating to patient identity—a patient's name, social security number, or phone number. In other implementations, generating the unique patient identifier independently of patient identify also includes: generating unique patient identifier with information relating to patient identity, as long as the unique patient identifier generated cannot be used (e.g., by an unauthorized third party) to deduce the patient's identity. For example, in some implementations, the unique patient identifier is generated by applying an irreversible hash function to a patient's date of birth, social security number, or a combination thereof.

In some implementations, the server stores (708) information identifying the patient identity and the unique patient identifier in a table or database (e.g., the server lookup table 158). More detail on the server lookup table 158 is explained with reference to FIG. 5A. In some implementations, information identifying the patient identity includes a patient's name, home or work address, date of birth, phone number, or social security number.

In some implementations, after processing the request for a unique patient identifier, the server 106 receives (712) from a hyperspectral imaging device (e.g., the imaging device 104), a hyperspectral image data set. In some implementations, the hyperspectral image data set includes (714) a plurality of sub-images of an object, and an associated unique patient identifier.

In some implementations, each sub-image is obtained independently from other sub-images in the plurality of sub-images by: (i) illuminating the object using a light source, the light source being configured to emit light of a predefined wavelength or wavelength band; and (ii) collecting, from the object, backscattering of the predefined wavelength or wavelength band of the light emitted by the light source. For example, in some implementations, the imaging device 104 includes two or more plates (e.g., CCD imaging detectors), and backscattering corresponding to the sub-image 1 is collected by plate 1, while backscattering corresponding to the sub-image 2 is collected by plate 2. In some implementations, sub-image 1 is obtained at least in part concurrently (e.g., in parallel) with sub-image 2. In other implementations, sub-images 1 and 2 are obtained in a predefined sequence (e.g., based on each sub-image's corresponding wavelength or wavelength bands). In some implementations, the imaging device 104 illuminates the object and collects the backscattering for a predefined number of iterations, with light of same or different wavelengths or wavelength bands, to generate the plurality of sub-images.

In some implementations, an exposure time for each respective sub-image in the plurality of sub-images is determined in accordance with the wavelength or wavelength band of the illumination. For example, exposure time for sub-images obtained using a light of narrow wavelength band (e.g., 10-50 nm) is shorter than that for sub-images obtained using a light of broad wavelength (e.g., 50-100 nm). In some implementations, the exposure time for each sub-image in the plurality of sub-images is different, for example, when each sub-image in the plurality of sub-images is obtained using light of different wavelengths or wavelength bands.

In some implementations, each sub-image in the plurality of sub-images is resolved at different times. For example, in implementations where the imaging device 104 includes two or more plates, backscattering of light of different wavelength or wavelength band is collected by different plates, and resolved at differ timings (e.g., within a predefined time of their respective collections). For example, sub-image 1 (416-1 in FIG. 4) is resolved 1 ms after corresponding backscattering is collected, at which time sub-image 2 (416-2 in FIG. 4) is still being generated.

In some implementations, each sub-image in the plurality of sub-images is transmitted to the server at a different time. For example, the sub-image 1 (416-1 in FIG. 4) is transmitted to the server 106 within 1 millisecond of its resolution, at which time sub-image 2 (416-2 in FIG. 4) is still being generated by the imaging device 104, or vice versa.

In some implementations, the hyperspectral image data set received by the server is encrypted using one or more encryption algorithms (e.g., to guard against unauthorized access by a third part, such as an identity thief). In some implementations, one or more encryption algorithms include encryption algorithms employing public key or private key, such as RSA.

In some implementations, the hyperspectral image data set is compressed (e.g., to reduce network traffic). In some implementations, the hyperspectral image data set is compressed using one or more image compression algorithms, such as Huffman coding, arithmetic coding, and LZW. In some implementations, the one or more image compression algorithms include lossless compression algorithms or lossy compression algorithms. In other implementations, the hyperspectral image data set is not compressed (e.g., to reduce response time by the imaging device 104). In some embodiments, the one or more image compression algorithms include vector quantization (VQ) based algorithms such as mean-normalized vector quantization (M-NVQ). See Ryan and Arnold, “The lossless compression of AVIRIS images by vector quantization,” IEEE Trans. Geoscience and Remote Sensing, Vol. 35, No. 3, May 1997, which is hereby incorporated herein by reference. In some embodiments, the one or more image compression algorithms include VQ based algorithms that involve locally optimal design of a partitioned vector quantizer for the encoding of source vectors drawn from hyperspectral images. See Motta et al., “Compression of hyperspectral imagery,” Data Compression Conference. Proceedings. DCC 2003, pp. 25-27, March 2003 which is hereby incorporated herein by reference. In some embodiments, the one or more image compression algorithms include spatial M-NVQ techniques that jointly optimized the spatial M-NVQ and spectral Discrete Cosine Transform (DCT) to produce compression ratios significantly better than those obtained by the optimized spatial M-NVQ technique alone. See Pickering and Ryan, “Efficient spatial-spectral compression of hyperspectral data,” IEEE Trans. Geoscience and Remote Sensing, 39, No. 7, July 2001, which is hereby incorporated herein by reference. In some embodiments, the one or more image compression algorithms include wavelet transform algorithms such as Shapiro's embedded zerotree wavelet, set partitioning in hierarchical trees, and set partitioned embedded bloCK. See Shapiro, “Embedded image coding using zerotrees of wavelet coefficients,” IEEE Trans. Signal Processing, vol. 41, pp. 3445-3462, December 1993, Said and Pearlman, “A new, fast and efficient image codec based on set partitioning in hierarchical trees,” IEEE Trans. on Circuits and Systems for Video Technology 6, pp. 243-250, June 1996, and Islam and Pearlman, “An embedded and efficient low-complexity hierarchical image coder,” in Proc. SPIE Visual Comm. and Image Processing, vol. 3653, pp. 294-305, 1999.

In some implementations, after receiving the hyperspectral image data set, the server forms (716) a hyperspectral image in accordance therewith. In some implementations, forming the hyperspectral image using the hyperspectral image data set includes: (i) assembling the plurality of sub-images into a spectral hypercube and (ii) processing the spectral hypercube to generate the hyperspectral image.

In some implementations, after forming the hyperspectral image data set, the server 106 identifies a client computer 102 among a plurality of client computers, using the unique patient identifier included in or associated with the hyperspectral image data set, and the server lookup table. The server 106 then transmits (720) the hyperspectral image to the first client computer (e.g., for a physician to review).

In some implementations, the server 106 transmits the hyperspectral image to the first client computer via a wireless network. For example, the client 102 and the server 106 are wirelessly connected via a wireless network provided by a hospital or a doctor's office where the patient is being treated, or by an independent service provider, such as AT&T, Sprint, or T-Mobile.

In some implementations, the server 106 and the hyperspectral imaging device 104 are connected through a local network. For example, the imaging device 104 is a portable device, which a physician carries to different locations of a hospital, and imaging data can be transmitted between the imaging device 104 and the server 106, through a local network, wired or wireless, within the hospital.

In some implementations, the first client computer 102 and the server 104 are within the same local network or protected by the same firewall. For example, the client 102 and the server 106 are within a same LAN within a hospital, a clinic, or a doctor's office, where the patient is being treated. For another example, the client 102 and the server 106 are behind a same firewall, which protects data transmission between the client 102 and the server 106 from unauthorized interception.

In some implementations, the server and the first client computer are two distinct computing devices (e.g., two different computers) connected through a computer network (e.g., the communication network 108 in FIG. 1). In other implementations, the server (e.g., the server 106) is a component of the first client computer. In some embodiments, the server 106 is a software module (e.g., programming packages) on the client 102 (See FIG. 2). In other implementations, the server is a hardware module (e.g., IC chipsets) on the first client computer.

In some implementations, the server (e.g., the server 106), with or without human intervention, further compares the hyperspectral image data set to one or more hyperspectral signatures, which correspond to one or more medical conditions. In some implementations, in accordance with the comparison, the server identifies a medical condition when the hyperspectral image data set and a hyperspectral signature share a predefined degree of similarity.

In some implementations, after forming the hyperspectral image (e.g., the hyperspectral image 105 in FIG. 1), the server (e.g., the server 106), with or without human intervention, further compares the hyperspectral image to one or more hyperspectral signatures, which correspond to one or more medical conditions. In some implementations, in accordance with the comparison, the server identifies a medical condition when the hyperspectral image and a hyperspectral signature share a predefined degree of similarity. In other implementations, the server transmits the hyperspectral image to the first client computer (e.g., the client 102-A), which compares of the hyperspectral image to the hyperspectral signatures, and identifies one or more medical conditions in accordance with the comparison.

In some implementations, based on the hyperspectral image, and optionally, one or more medical conditions identified, the treating physician diagnoses the patient and advises the patient regarding treatment, if necessary.

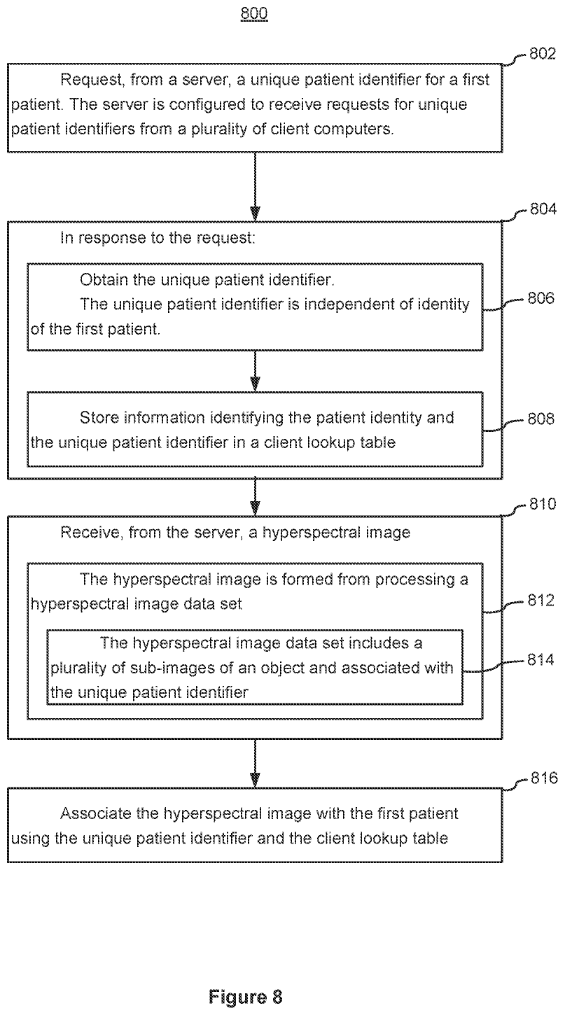

FIG. 8 is a flowchart illustrating a method for processing hyperspectral image via computer network, at a client, in accordance with some implementations.

In some implementations, a first client computer (e.g., the client 102-A) requests (802), from a server (e.g. the server 106-A), a unique patient identifier for a first patient. In some implementations, the server is configured to receive requests for unique patient identifiers from a plurality of client computers (e.g., the client 102-A . . . the client 102-N). For example, the server 106-A, a server located in a hospital, is communicatively connected with and receives requests for patient identifiers from two or more computers (e.g., computers that belong to different physicians or are located at different locations).

In some implementations, in response to the request (804), the first client computer obtains (806) the unique patient identifier from the server. In some implementations, the unique patient identifier is generated independently of the identity of the first patient. In some implementations, generating the unique patient identifier independently of patient identify includes: generating the unique patient identifier without reference to information relating to patient identity—a patient's name, social security number, or phone number. In other implementations, generating the unique patient identifier independently of patient identity also includes: generating unique patient identifier by applying a reverse-engineering proof function to information relating to patient identity, so that the unique patient identifier generated cannot be used to by an unauthorized third party to deduce the patient's identity. For example, in some implementations, the unique patient identifier is generated by applying an irreversible hash function to a patient's date of birth, social security number, or a combination thereof.

In some implementations, the first client computer stores (808) information identifying the patient identity and the unique patient identifier in a client lookup table (e.g., a table or a database). More detail on the client lookup table 112 is explained with reference to FIG. 5B. In some implementations, information identifying the patient identity includes a patient's name, home or work address, date of birth, phone number, or social security number.

In some implementations, the first client computer receives (810), from the server, a hyperspectral image (e.g., the hyperspectral image 105). In some implementations, the hyperspectral image is formed (812), by the server or the imaging device 104, from processing a hyperspectral image data set. In some implementations, the hyperspectral image data set includes (814) a plurality of sub-images of an object and an associated patient identifier. In some implementations, forming the hyperspectral image using the hyperspectral image data set includes: (i) assembling the plurality of sub-images into a spectral hypercube, and (ii) processing the spectral hypercube to generate the hyperspectral image.

In some implementations, after receiving the hyperspectral image, the first client computer associates (812) the hyperspectral image with the first patient using the unique patient identifier and the client lookup table.

In some implementations, each sub-image is obtained independently from other sub-images in the plurality of sub-images by: (i) illuminating the object using a light source, the light source being configured to emit light of a predefined wavelength or wavelength band, and (ii) collecting, from the object, backscattering of the predefined wavelength or wavelength band of the light emitted by the light source. For example, in implementations where the imaging device 104 includes two or more plates, and backscattering corresponding to the sub-image 1 is collected by plate 1, while backscattering corresponding to the sub-image 2 is collected by plate 2. In some implementations, sub-image 1 is obtained at least in part concurrently (e.g., in parallel) with sub-image 2. In other implementations, sub-images 1 and 2 are obtained in predefined sequence (e.g., based on each sub-image's corresponding wavelength or wavelength bands). In some implementations, the imaging device 104 illuminates the object and collects the backscattering for a predefined number of iterations, with light of same or different wavelengths or wavelength bands, to generate the plurality of sub-images.

In some implementations, an exposure time for each respective sub-image in the plurality of sub-images is determined in accordance with the wavelength or wavelength band of the illumination. For example, exposure time for sub-images obtained using a light of narrow wavelength band (e.g., 10-50 nm) is shorter than that for sub-images obtained using a light of broad wavelength (e.g., 50-100 nm). In some implementations, the exposure time for each sub-image in the plurality of sub-images is different, for example, when each sub-image in the plurality of sub-images is obtained using light of different wavelengths or wavelength bands.

In some implementations, each sub-image in the plurality of sub-images is resolved at different timings. For example, in implementations where the imaging device 104 includes two or more plates, backscattering of light of different wavelength or wavelength band is collected by different plates, and resolved at differ timings (e.g., within a predefined time of their respective collections). For example, the sub-image 1 (416-1 in FIG. 4) is resolved 1 ms after corresponding backscattering is collected, at which time the sub-image 2 (416-2 in FIG. 4) is still being generated.

In some implementations, each sub-image in the plurality of sub-images is transmitted to the first client computer at a different time. For example, in implementations where the client 102 (rather than the server 106) forms the hyperspectral image using the hyperspectral image data set, the sub-image 1 (416-1 in FIG. 4) is transmitted to the client 102, while the sub-image 2 (416-2 in FIG. 4) is still being generated by the imaging device 104.

In some implementations, the hyperspectral image data set is encrypted using one or more encryption algorithms (e.g., to guard against unauthorized access by a third part, such as an identity thief). In some implementations, one or more encryption algorithms include encryption algorithms employing public key or private key, such as, RSA.

In some implementations, the hyperspectral image data set is compressed (e.g., to reduce network traffic). In some implementations, the hyperspectral image data set is compressed using one or more image compression algorithms, such as Huffman coding, arithmetic coding, and LZW. In some implementations, the one or more image compression algorithms include lossless compression algorithms or lossy compression algorithms. In other implementations, the hyperspectral image data set is not compressed (e.g., to reduce response time by the imaging device 104). In some embodiments, the one or more image compression algorithms include vector quantization (VQ) based algorithms such as mean-normalized vector quantization (M-NVQ). See Ryan and Arnold, “The lossless compression of AVIRIS images by vector quantization,” IEEE Trans. Geoscience and Remote Sensing, Vol. 35, No. 3, May 1997, which is hereby incorporated herein by reference. In some embodiments, the one or more image compression algorithms include VQ based algorithms that involve locally optimal design of a partitioned vector quantizer for the encoding of source vectors drawn from hyperspectral images. See Motta et al., “Compression of hyperspectral imagery,” Data Compression Conference. Proceedings. DCC 2003, pp. 25-27, March 2003 which is hereby incorporated herein by reference. In some embodiments, the one or more image compression algorithms include spatial M-NVQ techniques that jointly optimized the spatial M-NVQ and spectral Discrete Cosine Transform (DCT) to produce compression ratios significantly better than those obtained by the optimized spatial M-NVQ technique alone. See Pickering and Ryan, “Efficient spatial-spectral compression of hyperspectral data,” IEEE Trans. Geoscience and Remote Sensing, 39, No. 7, July 2001, which is hereby incorporated herein by reference. In some embodiments, the one or more image compression algorithms include wavelet transform algorithms such as Shapiro's embedded zerotree wavelet, set partitioning in hierarchical trees, and set partitioned embedded bloCK. See Shapiro, “Embedded image coding using zerotrees of wavelet coefficients,” IEEE Trans. Signal Processing, vol. 41, pp. 3445-3462, December 1993, Said and Pearlman, “A new, fast and efficient image codec based on set partitioning in hierarchical trees,” IEEE Trans. on Circuits and Systems for Video Technology 6, pp. 243-250, June 1996, and Islam and Pearlman, “An embedded and efficient low-complexity hierarchical image coder,” in Proc. SPIE Visual Comm. and Image Processing, vol. 3653, pp. 294-305, 1999.

In some implementations, the first client computer receives the hyperspectral image from the server via a wireless network. For example, the client 102 and the server 106 are wirelessly connected. In some implementations, the first client computer is connected with the server via a local wireless network (e.g., provided by a hospital associated with the treating physician, or by an independent service provider, such as AT&T, Sprint, or T-Mobile).

In some implementations, the first client computer and the server are within the same local network or protected by the same firewall. For example, the client 102 and the server 106 are within a same LAN within a hospital, a clinic, or a doctor's office, where the patient is being treated.

In some implementations, the first client computer and the server are within the same local network or protected by the same firewall. For example, the client 102 and the server 106 are within a same LAN within a hospital, a clinic, or a doctor's office, where the patient is being treated. In another example, the client 102 and the server 106 are behind a common firewall, which protects data transmission between the client 102 and the server 106 from unauthorized interception.

In some implementations, the first client computer and the server are two distinct computing devices connected through a computer network (e.g., the communication network 108 in FIG. 1). In other implementations, the server (e.g., the server 106) is a component of the first client computer. In some embodiments, the server 106 is a software module (e.g., programming packages) on the client 102 (See FIG. 2). In other implementations, the server is a hardware module (e.g., IC chipsets) on the first client computer.

In some implementations, after obtaining the hyperspectral image (e.g., the hyperspectral image 105 in FIG. 1), the first client computer (e.g., the server 106), with or without human intervention, further compares the hyperspectral image with one or more hyperspectral signatures, which correspond to one or more medical conditions. In some implementations, in accordance with the comparison, the first client computer identifies a medical condition when the hyperspectral image and a hyperspectral signature share a predefined degree of similarity.

In some implementations, based on the hyperspectral image, and optionally, one or more medical conditions identified based on the comparison, the treating physician diagnoses the patient, and advises the patient on treatment if necessary.

The methods illustrated in FIGS. 6, 7, and 8 may be governed by instructions that are stored in a computer readable storage medium and that are executed by at least one processor of at least one host device (or at least one server). Each of the operations shown in FIGS. 6, 7, and 8 may correspond to instructions stored in a non-transitory computer memory or computer readable storage medium. In various implementations, the non-transitory computer readable storage medium includes a magnetic or optical disk storage device, solid state storage devices such as Flash memory, or other non-volatile memory device or devices. The computer readable instructions stored on the non-transitory computer readable storage medium may be in source code, assembly language code, object code, or other instruction format that is interpreted and/or executable by one or more processors.

Plural instances may be provided for components, operations or structures described herein as a single instance. Finally, boundaries between various components, operations, and data stores are somewhat arbitrary, and particular operations are illustrated in the context of specific illustrative configurations. Other allocations of functionality are envisioned and may fall within the scope of the implementation(s). In general, structures and functionality presented as separate components in the example configurations may be implemented as a combined structure or component. Similarly, structures and functionality presented as a single component may be implemented as separate components. These and other variations, modifications, additions, and improvements fall within the scope of the implementation(s).

As used herein, hyperspectral and multispectral imaging are related techniques in larger class of spectroscopy commonly referred to as spectral imaging or spectral analysis. Typically, hyperspectral imaging relates to the acquisition of a plurality of images, each image representing a narrow spectral band collected over a continuous spectral range, for example, 20 spectral bands having a FWHM bandwidth of 20 nm each, covering from 400 nm to 800 nm. In contrast, multispectral imaging relates to the acquisition of a plurality of images, each image representing a narrow spectral band collected over a discontinuous spectral range. For the purposes of the present disclosure, the terms “hyperspectral” and “multispectral” are used interchangeably and refer to a plurality of images, each image representing a narrow spectral band (having a FWHM bandwidth of between 10 nm and 30 nm, between 5 nm and 15 nm, between 5 nm and 50 nm, less than 100 nm, between 1 and 100 nm, etc.), whether collected over a continuous or discontinuous spectral range.

It will also be understood that, although the terms “first,” “second,” etc. may be used herein to describe various elements, these elements should not be limited by these terms. These terms are only used to distinguish one element from another. For example, a first client computer could be termed a second client computer, and, similarly, a second client computer could be termed a first client computer, which changing the meaning of the description, so long as all occurrences of the “first client computer” are renamed consistently and all occurrences of the “second client computer” are renamed consistently. The first client computer, and the second client computer are both client computers, but they are not the same client computer.

The terminology used herein is for the purpose of describing particular implementations only and is not intended to be limiting of the claims. As used in the description of the implementations and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined (that a stated condition precedent is true)” or “if (a stated condition precedent is true)” or “when (a stated condition precedent is true)” may be construed to mean “upon determining” or “in response to determining” or “in accordance with a determination” or “upon detecting” or “in response to detecting” that the stated condition precedent is true, depending on the context.

The foregoing description included example systems, methods, techniques, instruction sequences, and computing machine program products that embody illustrative implementations. For purposes of explanation, numerous specific details were set forth in order to provide an understanding of various implementations of the inventive subject matter. It will be evident, however, to those skilled in the art that implementations of the inventive subject matter may be practiced without these specific details. In general, well-known instruction instances, protocols, structures and techniques have not been shown in detail.

The foregoing description, for purpose of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

1. A method comprising:
at a server:
(A) obtaining, from a first client computer in a plurality of client computers, a request for a unique patient identifier;
(B) in response to the request,
generating the unique patient identifier independent of patient identity;
storing the unique patient identifier and information identifying the first client computer in a server lookup table; and
transmitting the unique patient identifier to the first client computer;
(C) receiving, from a hyperspectral imaging device, a hyperspectral image data set, the hyperspectral image data set including a plurality of sub-images of an object and the unique patient identifier, wherein the unique patient identifier is associated with the plurality of sub-images;
(D) forming a processed hyperspectral image using the hyperspectral image data set;
(E) identifying the first client computer in the plurality of client computers using the unique patient identifier included in the hyperspectral image data set, and the server lookup table; and
(F) transmitting, to the first client computer, the processed hyperspectral image.

2. The method of claim 1, wherein each respective sub-image in the plurality of sub-images is obtained independently from other sub-images in the plurality of sub-images by:
illuminating the object using a light source, the light source being configured to emit light of a predefined wavelength or wavelength band; and
collecting, from the object, backscattering of the predefined wavelength or wavelength band of the light emitted by the light source.

3. The method of claim 1, wherein an exposure time for each respective sub-image in the plurality of sub-images is determined using the wavelength or wavelength band corresponding to the respective sub-image.

4. The method of claim 1, wherein the exposure time for each respective sub-image in the plurality of sub-images is different.

5. The method of claim 1, wherein each respective sub-image in the plurality of sub-images is resolved at a different time.

6. The method of claim 1 wherein each respective sub-image in the plurality of sub-images is received by the server at a different time.

7. The method of claim 1, wherein the hyperspectral image data set is encrypted at a time when it is received from the hyperspectral imaging device.

8. The method of claim 1 wherein the transmitting (F) comprises transmitting the processed hyperspectral image to the first client computer via a wireless network.

9. The method of claim 1 wherein the server and the hyperspectral imaging device are connected through a local network.

10. The method of claim 1, wherein the first client computer and the server are within the same local network or protected by a common firewall.

11. The method of claim 1, wherein the server is a software module on the first client computer.

12. The method of claim 1, wherein forming the processed hyperspectral image using the hyperspectral image data set includes:
assembling the plurality of sub-images into a spectral hypercube; and
processing the spectral hypercube to generate the processed hyperspectral image.

13. The method of claim 1, further comprising:
comparing the processed hyperspectral image to one or more hyperspectral signatures, the one or more hyperspectral signatures corresponding to one or more medical conditions; and
identifying a likelihood, probability or identity of a medical condition associated with the patient corresponding to the processed hyperspectral image when the processed hyperspectral image is deemed similar to a hyperspectral signature in the one or more hyperspectral signatures.

14. The method of claim 1, further comprising:
comparing the hyperspectral image data set to one or more hyperspectral signatures, the one or more hyperspectral signatures corresponding to one or more medical conditions; and
identifying a likelihood, probability or identity of a medical condition associated with the patient corresponding to the processed hyperspectral image when the processed hyperspectral image data set is similar to a hyperspectral signature in the one or more hyperspectral signatures.

15-27. (canceled)

28. A server system, comprising:
one or more processors;
memory; and
one or more programs, wherein the one or more programs are stored in the memory and are configured to be executed by the one or more processors, the one or more programs including instructions for:
(A) obtaining, from a first client computer in a plurality of client computers, a request for a unique patient identifier;
(B) in response to the request,
generating the unique patient identifier independent of patient identity;
storing the unique patient identifier and information identifying the first client computer in a server lookup table; and
transmitting the unique patient identifier to the first client computer;
(C) receiving, from a hyperspectral imaging device, a hyperspectral image data set, the hyperspectral image data set including a plurality of sub-images of an object and the unique patient identifier, wherein the unique patient identifier is associated with the plurality of sub-images;
(D) forming a processed hyperspectral image using the hyperspectral image data set;
(E) identifying the first client computer in the plurality of client computers using the unique patient identifier included in the hyperspectral image data set, and the server lookup table; and
(F) transmitting, to the first client computer, the processed hyperspectral image.

29. (canceled)

30. A non-transitory computer readable storage medium storing one or more programs, the one or more programs comprising instructions, which when executed by a computer system with one or more processors, cause the computer system to:
(A) obtain, from a first client computer in a plurality of client computers, a request for a unique patient identifier;
(B) in response to the request,
generate the unique patient identifier independent of patient identity;
store the unique patient identifier and information identifying the first client computer in a server lookup table; and
transmit the unique patient identifier to the first client computer;
(C) receive, from a hyperspectral imaging device, a hyperspectral image data set, the hyperspectral image data set including a plurality of sub-images of an object and the unique patient identifier, wherein the unique patient identifier is associated with the plurality of sub-images;
(D) form a processed hyperspectral image using the hyperspectral image data set;
(E) identify the first client computer in the plurality of client computers using the unique patient identifier included in the hyperspectral image data set, and the server lookup table; and
(F) transmit, to the first client computer, the processed hyperspectral image.

31-44. (canceled)
